(19) United States
(12) Patent Application Publication
(10) Pub. No.: US 2026/0330631 A1
(43) Pub. Date: Oct. 22, 2026

(54) INFORMATION PROCESSING DEVICE, ARRHYTHMIA DETECTION DEVICE, REGION IDENTIFICATION METHOD, AND COMPUTER-READABLE STORAGE MEDIUM

(71) Applicant: Kanadevia Corporation, Osaka-shi, Osaka (JP)

(72) Inventors: Hirosuke MIKI, Osaka-shi, Osaka (JP); Masahiro TOMARU, Osaka-shi, Osaka (JP)

(21) Appl. No.: 19/483,887

(22) PCT Filed: Mar. 1, 2024
(86) PCT No.: PCT/JP2024/007721

§ 371 Date: Nov. 12, 2025

(30) Foreign Application Priority Data
May 17, 2023 (JP) 2023-081782

(51) Int. Cl. G06T 7/00; G06T 5/50; G06T 7/11; G06V 10/25; G06V 10/764; G16H 30/40

(52) U.S. Cl. CPC G06T 7/0012; G06T 5/50; G06T 7/11; G06V 10/25; G06V 10/764; G16H 30/40; G06T 2207/10132; G06T 2207/20221; G06T 2207/30048

(57) ABSTRACT

In order to enhance precision in detection of a detection target region from an input image obtained by image-capturing of a target object, an information processing device includes: a detection section that detects, from an input image obtained by image-capturing of a target object, target regions included in the target object and aggregated regions each constituted by two or more of the target regions, the detection being carried out by using a learning model having been trained using a training image in which the target regions and the aggregated regions are given with respective different classifications as annotations; and an identifying section that identifies a detection missed region in the aggregated regions having been detected, the detection missed region being a target region which has not been detected.

### TECHNICAL FIELD

The present invention relates to an information processing device and the like each of which detects a target region included in a target object from an input image obtained by image-capturing of the target object.

### BACKGROUND ART

A technology for detecting a detection target region from an input image obtained by image-capturing of a target object is widely used. For example, Patent Literature 1 discloses an ultrasound diagnostic device suitable for diagnosis or measurement of a fetus's heart. With this ultrasound diagnostic device, a region corresponding to a portion-of-interest in the fetus's heart is extracted from echo data for forming a tomographic image which echo data is obtained via transmission/reception of ultrasound waves.

### CITATION LIST

### Patent Literatures

### Patent Literature 1

Japanese Patent No. 4206107

### SUMMARY OF INVENTION

### Technical Problem

However, depending on the target object and the input image, it is difficult to always detect a detection target region appropriately. In some cases, detection may be missed.

An aspect of the present invention has an object to enhance precision in detection of a detection target region from an input image obtained by image-capturing of a target object.

### Solution to Problem

In order to attain the above object, an information processing device in accordance with an aspect of the present invention includes: a detection section that detects, from an input image obtained by image-capturing of a target object, target regions included in the target object and aggregated regions each constituted by two or more of the target regions, the detection being carried out by using a learning model having been trained using a training image in which the target regions and the aggregated regions are given with respective different classifications as annotations; and an identifying section that identifies a detection missed region in the aggregated regions having been detected, the detection missed region being a target region which has not been detected.

A region identification method in accordance with an aspect of the present invention is a region identification method that is executed by one or more information processing devices, the region identification method including: a detection step of detecting, from an input image obtained by image-capturing of a target object, target regions included in the target object and aggregated regions each constituted by two or more of the target regions, the detection being carried out by using a learning model having been trained using a training image in which the target regions and the aggregated regions are given with respective different classifications as annotations; and an identification step of identifying a detection missed region in the aggregated regions having been detected, the detection missed region being a target region which has not been detected.

An information processing device in accordance with each aspect of the present invention may be realized by a computer. In this case, the present invention also encompasses: a control program for causing the computer to function as each of the sections (software elements) included in the information processing device so that the information processing device is realized by the computer; and a computer-readable storage medium in which the control program is stored.

### Advantageous Effects of Invention

In accordance with an aspect of the present invention, it is possible to enhance precision in detection of a detection target region from an input image obtained by image-capturing of a target object.

### BRIEF DESCRIPTION OF DRAWINGS

FIG. 1 is a view illustrating an example of a configuration of an information processing system in accordance with an embodiment of the present invention.

FIG. 2 is a block diagram illustrating one example of a configuration of a main part of a learning device in accordance with an embodiment of the present invention.

FIG. 3 is a block diagram illustrating one example of a configuration of a main part of an information processing device in accordance with an embodiment of the present invention.

FIG. 4 is a view illustrating an example of target regions in an image obtained by image-capturing of a heart.

FIG. 5 is a view illustrating examples of aggregated regions in the image obtained by image-capturing of the heart.

FIG. 6 is a view illustrating one example of a detection missed region in the image obtained by image-capturing of the heart.

FIG. 7 is a view illustrating one example of a trouble which may occur in a case where a detection missed region is identified on the basis of the target regions and the aggregated regions having been detected.

FIG. 8 is a view illustrating one example of a method for identifying a detection missed region on the basis of the target regions and the aggregated regions having been detected.

FIG. 9 is a view illustrating another example of a method for identifying a detection missed region on the basis of the target regions and the aggregated regions having been detected.

FIG. 10 is a view illustrating one example of an expansion process executed by an identifying section.

FIG. 11 is a flowchart illustrating one example of a flow of a process executed by an information processing device.

FIG. 12 is a block diagram illustrating one example of a configuration of a main part of an information processing device in accordance with another embodiment of the present invention.

FIG. 13 is a flowchart illustrating one example of a flow of a process executed by the information processing device.

FIG. 14 is a view illustrating a positional relation between the target regions in the image obtained by image-capturing of the heart.

FIG. 15 is a view illustrating an example of a configuration of an information processing system including an arrhythmia detection device in accordance with an embodiment of the present invention.

FIG. 16 is a view illustrating one example of a process executed by the information processing system including the arrhythmia detection device.

FIG. 17 is a view illustrating one example of a process in which the arrhythmia detection device detects arrhythmia.

FIG. 18 is a flowchart illustrating one example of a process executed by the information processing system.

FIG. 19 is a view illustrating an example of target regions in an image of a dragon fly's head, captured from front of the dragon fly's head.

FIG. 20 is a view illustrating an example of aggregated regions in the image of the dragon fly's head, captured from the front of the dragon fly's head.

### DESCRIPTION OF EMBODIMENT

### First Embodiment

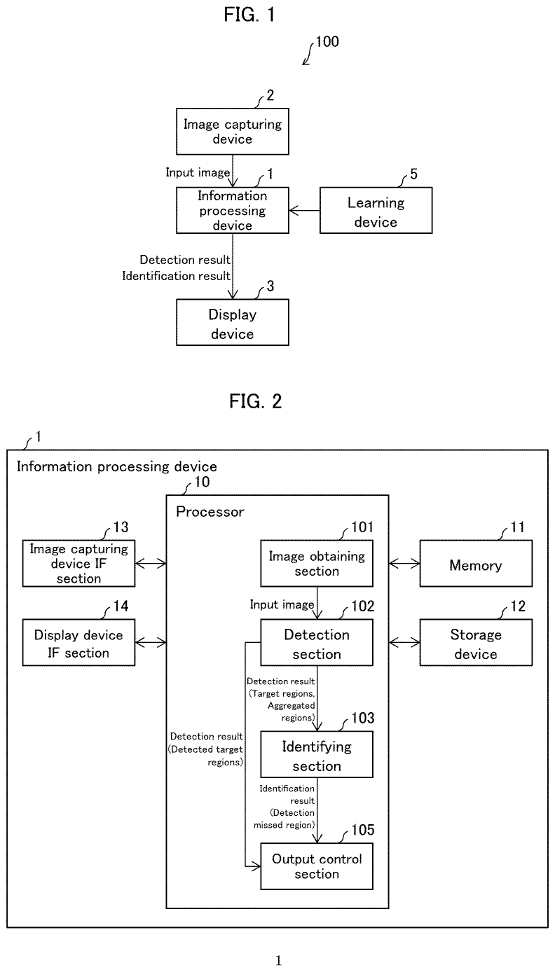

The following will describe, with reference to FIGS. 1 to 11, details of an embodiment of the present invention.

### Overview of Information Processing Device 1

An information processing device 1 in accordance with an embodiment of the present invention detects, from an input image obtained by image-capturing of a target object, target regions included in the target object and aggregated regions each constituted by two or more of the target regions. The detection of the target regions and the aggregated regions is carried out by using a learning model trained using a training image in which the target regions and the aggregated regions are given with respective different classifications as annotations. Then, by using a result of the detection of the aggregated regions and the target regions, the information processing device 1 identifies a detection missed region in the aggregated regions having been identified, the detection missed region being a target region which has not been detected.

In a case of using a learning model having been trained using a training image in which only the target regions of the target object are given with respective different classifications as annotations, one of or some of the target regions may possibly be failed to be detected. The inventors of the present invention found a detection method that uses a learning model trained using a training image in which not only the target regions but also the aggregated regions are given with respective different classifications as annotations. Even if detection of one of or some of the target regions is missed, use of the above detection method makes it possible to identify, in the detected aggregated regions, the target region(s) having not been detected. This can enhance precision in detection of the target regions.

A target object to which the present invention is applicable only needs to include a plurality of regions which can be target regions. Thus, there is no particular limitation on the target object. For example, the target object may be an organ of an animal. A heart of a mammal, including a human, has left and right atriums and left and right ventricles. In evaluation of a function of a heart, the atriums and ventricles can be detection targets (i.e., target regions). In this case, the input image may be a medical image, and may be, for example, an ultrasonic image (e.g., an echo image for forming a tomographic image). The ultrasonic image may be a moving image including images of a plurality of frames.

The target object is not limited to a heart, and may alternatively be a lung, a liver, or the like. Each of the lung and the liver has a plurality of lobes, and the lobes can be target regions. In this case, the input image may be an X-ray computed tomography (CT) image.

The target object is not limited to an organ of an animal, and may alternatively be any kind of living organism, including an insect, a plant, and the like. For example, an insect's head includes a plurality of sensory organs which can be detection targets. Further, a cell colony constituting a living organism's characteristic organ or tissue (e.g., a stoma, a vascular bundle, or the like of a plant) can be a target region.

The target object is not limited to a living organism, but may be a non-biological object. For example, the target object may be an automobile. In this case, regions corresponding to respective parts constituting the automobile can be target regions. Examples of the parts may include a windshield, a window glass of a door, a side mirror, a door, a wheel, and a tire. In this case, a combination of the windshield and the window glass of the door, a combination of the side mirror and the door, a combination of a plurality of doors (e.g., front and rear doors), a combination of the wheel and the tire, and/or the like may be aggregated regions constituted by a plurality of target regions.

The following will provide an explanation by taking, as an example, a case where the target object is a heart and the input image is an ultrasonic image obtained by image-capturing of the heart. In the ultrasonic image obtained by image-capturing of the heart, two ventricles and two atriums of the heart are each shown as a closed space.

### Configuration of Information Processing System 100

First, the following will describe, with reference to FIG. 1, a configuration of an information processing system 100 in accordance with an embodiment of the present invention. FIG. 1 is a view illustrating an example of a configuration of the information processing system 100. The information processing system 100 may include an image capturing device 2, the information processing device 1, a display device 3, and a learning device 5.

The image capturing device 2 may be an ultrasonic imaging device capable of capturing an image of an organ of a subject in a non-invasive manner. As shown in FIG. 1, the image capturing device 2 may be communicably connected with the information processing device 1. In this case, the information processing device 1 may directly obtain, from the image capturing device 2, an input image obtained by image-capturing of the image capturing device 2. Note that the input image obtained by image-capturing carried out by the image capturing device 2 may be stored in a portable storage medium, for example. In this case, the information processing device 1 may read out the input image from the storage medium. Alternatively, the input image obtained by image-capturing carried out by the image capturing device 2 may be stored in a storage device (not illustrated) such that the input image is associated with subject information of the respective subject, for example. In this case, the information processing device 1 may obtain the input image from the storage device.

The display device 3 is a device capable of displaying various kinds of information output from the information processing device 1. The display device 3 may display, in addition to various kinds of information output from the information processing device 1, the input image, the subject information, and/or the like.

The information processing device 1 may be provided in a facility (e.g., a medical facility) in which the image capturing device 2 is provided or may be provided in a remote place. In a case where the information processing device 1 is provided in a remote place, the information processing device 1 may obtain the input image through communication carried out over a communication network such as the Internet.

Further, a part of the processes which are to be carried out by the information processing device 1 may be carried out by another computer system. For example, detection of the target regions and the aggregated regions, each of which is a detection target, is carried out through calculation using the later-described learning model. This calculation may be carried out by another computer system. In this case, the information processing device 1 may transmit information necessary to carry out the calculation to another computer system through communication carried out over a communication network and may receive a result of the calculation transmitted from the another computer system.

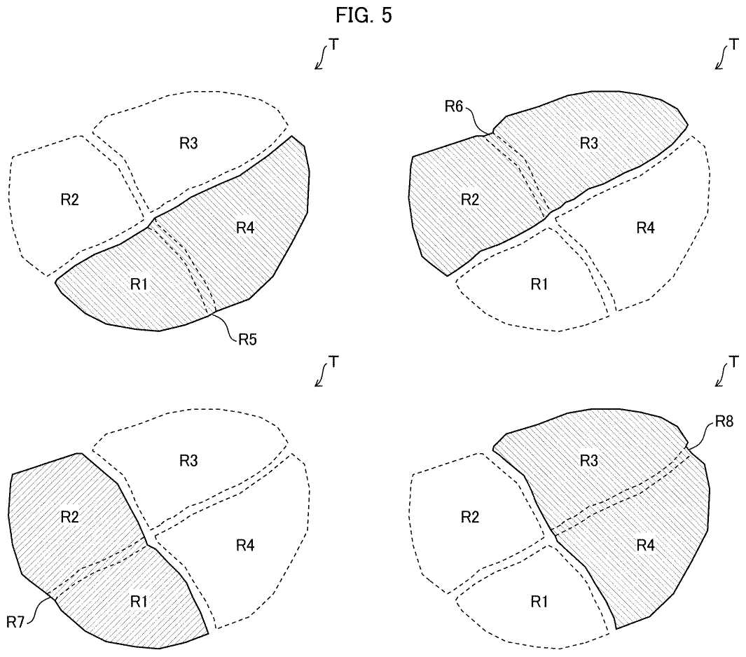

The learning device 5 generates, from the image of the heart captured by the image capturing device 2, a learning model used to detect the target regions and aggregated regions. The learning model may be the one generated by known machine learning. For example, the learning device 5 may generate a learning model such as a convolutional neural network. Each of the target regions may constitute at least one of the aggregated regions. By employing this configuration, all of the target regions can be subjects to be identified as detection missed regions. The target regions and the aggregated regions, each of which can be as a detection target, will be explained later using specific examples (see FIGS. 4 and 5).

The learning model generated by the learning device 5 is used to detect regions which are to be detected by the information processing device 1. The learning device 5 may be provided in the place where the information processing device 1 is provided, or may be provided in a place different from the place where the information processing device 1 is provided. The learning device 5 executes a program for causing a computer to function as the learning device. The function of the learning device 5 may be included in the information processing device 1. In this case, the learning device 5 is removed from the constituent elements of the information processing system 100.

### Configuration of Information Processing Device 1

Next, the following will describe a configuration of the information processing device 1 with reference to FIG. 2. FIG. 2 is a block diagram illustrating one example of a configuration of a main part of the information processing device 1. As shown in FIG. 2, the information processing device 1 includes a processor 10, a memory 11, and a storage device 12. The information processing device 1 may be, for example, a personal computer, a server, or a work station. The processor 10 loads an information processing program stored in the storage device 12 into the memory 11 and executes the information processing program, so that the processor 10 functions as each of the later-described sections, i.e., an image obtaining section 101 to an output control section 105.

The processor 10 can be realized by a logical circuit (hardware) formed in an integrated circuit (IC chip) or the like or can be realized by software. In a case where the processor 10 is realized by software, the processor 10 may be constituted by, for example, a Central Processing Unit (CPU), a Graphics Processing Unit (GPU), or a combination thereof. Further, in this case, the software is stored in the storage device 12. Then, the processor 10 loads the software into the memory 11 and executes the software.

Each of the memory 11 and the storage device 12 is a storage device in which various kinds of data used by the information processing device 1 is stored. The memory 11 is a storage device with which writing and reading of data can be carried out at a higher speed in comparison to the storage device 12. The storage device 12 has a larger data storage capacity in comparison to the memory 11. As the memory 11, a high-speed access memory such as a Synchronous Dynamic Random-Access Memory (SDRAM) can be employed, for example. As the storage device 12, a Hard Disk Drive (HDD), a Solid-State Drive (SSD), a Secure Digital (SD) card, an embedded Multi-Media Controller (eMMC), or the like can be employed, for example.

The information processing device 1 includes, as an interface (IF) with an external device, an image capturing device interface (IF) section 13 and a display device interface (IF) section 14. As each of these interfaces, any interface is applicable. Examples of such an interface may include a Universal Serial Bus (USB) and a High-Definition Multimedia Interface (HDMI) (registered trademark).

The image capturing device interface section 13 is an interface via which the image capturing device 2 and the information processing device 1 are communicably connected with each other. The image capturing device interface section 13 may be an interface for wired communication or an interface for radio communication. For example, a USB, a Local-Area Network (LAN), a wireless LAN, or the like can be employed as each of these interface sections. Each of these interfaces is an interface for obtaining the above-described input image from the image capturing device 2. The display device interface section 14 is an interface for transmitting, to the display device 3, information output from the information processing device

The display device interface section 14 is an interface which is used by the information processing device 1 to cause the non-illustrated display device 3 to display an image. For example, via the display device interface section 14, the information processing device 1 can cause the display device 3 to display an image obtained by image-capturing carried out by the image capturing device 2 and a detection result output from the information processing device 1. Note that the information processing device 1 may include a display section which can function as the display device 3.

The information processing device 1 may further include an interface via which the information processing device 1 communicates with various devices (e.g., a storage device) on a communication network such as the Internet, for example. Note that the information processing device 1 may include, in addition to the above-described interfaces, an interface and/or the like for access to, for example, an externally attached storage device (e.g., a hard disk).

As discussed above, the processor 10 functions as each of the image obtaining section 101, the detection section 102, the identifying section 103, and the output control section 105. The following will describe each of these sections.

The image obtaining section 101 obtains an input image obtained by image-capturing of a heart T which image-capturing is carried out by the image capturing device 2. There is no particular limitation on a method for obtaining the input image. For example, the image obtaining section 101 may obtain a captured image from the image capturing device 2 via the image capturing device interface section 13.

By using the learning model generated by machine learning, the detection section 102 detects target regions and aggregated regions from the input image obtained by image-capturing of the heart T. The machine learning for generating the learning model will be described later.

The identifying section 103 identifies a detection missed region in the detected aggregated regions, the detection missed region being a target region which has not been detected. For example, the identifying section 103 may identify, as the detection missed region, a region that is included in an overlapping region where some of the detected aggregated regions overlap each other and that has not been detected as a target region. Also in a case where the overlapping region includes two or more target regions and a part of these target regions has been detected, the identifying section 103 can identify a detection missed region. Alternatively, the identifying section 103 may identify, as the detection missed region, a region obtained by removing the detected target region(s) from the detected aggregated region(s).

The output control section 105 controls outputting of the detection result and the identification result given by the information processing device 1 to the display device 3.

With this configuration, the information processing device 1 can identify a detection missed region by a simple image process of removing a detected aggregated region from the detected aggregated regions or of detecting, as a target region, an overlapping region where the detected aggregated regions overlap each other. Details of a process in which the identifying section 103 identifies a detection missed region will be described later using specific examples.

### Configuration of Learning Device 5

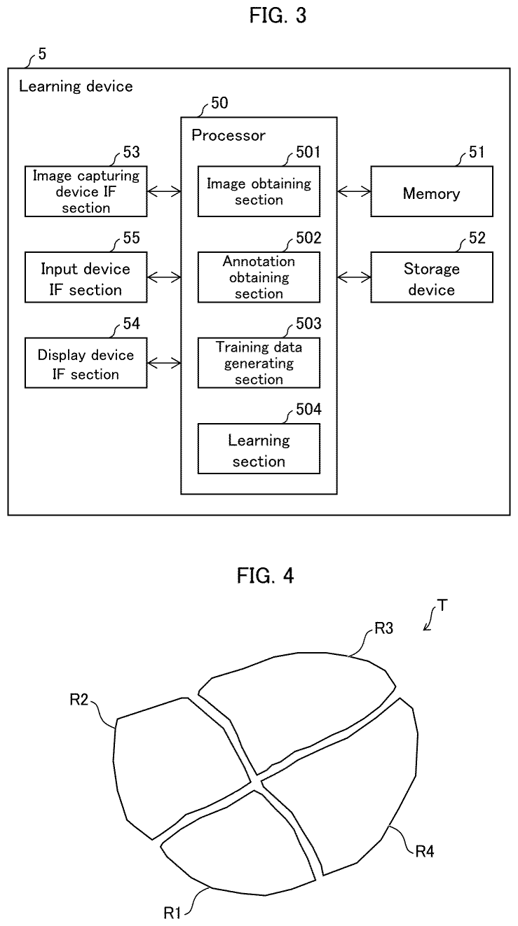

Next, the following will describe, with reference to FIG. 3, a configuration of the learning device 5 which generates the learning model. FIG. 3 is a block diagram illustrating one example of a configuration of a main part of the learning device 5. As shown in FIG. 3, the learning device 5 includes a processor 50, a memory 51, and a storage device 52. Similarly to the information processing device 1, the learning device 5 may be a personal computer, a server, or a work station. The processor 50 loads a learning program stored in the storage device 52 into the memory 51 and executes the information processing program, so that the processor 50 functions as each of the later-described sections, i.e., an image obtaining section 501 to a learning section 504.

The processor 50 can be realized by a logical circuit (hardware) formed in an integrated circuit (IC chip) or the like or can be realized by software. In a case where the processor 50 is realized by software, the processor 50 may be constituted by, for example, a CPU, a GPU, or a combination thereof. Further, in this case, the software is stored in the storage device 52. Then, the processor 50 loads the software into the memory 51 and executes the software.

Each of the memory 51 and the storage device 52 is a storage device in which various kinds of data used by the learning device 5 is stored. The memory 51 is a storage device with which writing and reading of data can be carried out at a higher speed in comparison to the storage device 52. The storage device 52 has a larger data storage capacity in comparison to the memory 51. As the memory 51, a high-speed access memory such as a SDRAM can be employed, for example. As the storage device 52, a HDD, a SSD, a SD card, an eMMC, or the like can be employed, for example.

Further, the learning device 5 includes, as an interface (IF) with an external device, an image capturing device interface section 53, an input device interface section 55, and a display device interface section 54. The input device interface section 55 is an interface which accepts an input signal from an input device such as a keyboard or a mouse. The input device interface section 55 may be an interface such as a USB, for example. Among the interfaces included in the learning device 5, an interface having the same name as that included in the information processing device 1 shown in FIG. 2 has the same function as that included in the information processing device 1. Therefore, description thereof will be omitted.

As discussed above, the processor 50 functions as each of the image obtaining section 501, the annotation obtaining section 502, the training data generating section 503, and the learning section 504. The following will describe each of the sections.

The image obtaining section 501 obtains an image for training (i.e., a training image) obtained by image-capturing carried out by the image capturing device 2. There is no particular limitation on a method for obtaining the training image. For example, the image obtaining section 501 may obtain the above-described training image from the image capturing device 2 via the image capturing device interface section 53. For example, by communication with the information processing device 1, the image obtaining section 501 may obtain the training image stored in the storage device 12 of the information processing device 1. For another example, in a case where the training image is stored in a storage device external to the information processing device 1, the image obtaining section 301 may access the storage device to obtain the captured image.

The annotation obtaining section 502 obtains the respective different classifications for the target regions and the aggregated regions in the heart T in the training image. The classifications can be input via the input device by a medical worker who has checked the training image.

### Target Regions R1 to R4 and Aggregated Regions R5 to R8

The description here will discuss, with reference to FIGS. 4 and 5, the target regions and the aggregated regions each including two or more target regions in the image obtained by image-capturing of the heart T. FIG. 4 is a view illustrating an example of target regions R1 to R4 in the image obtained by image-capturing of the heart T. FIG. 5 is a view illustrating an example of aggregated regions R5 to R8 in the image obtained by image-capturing of the heart T.

The heart T includes four anatomical regions (i.e., two ventricles and two atriums). Therefore, in the heart T, the target region R1 corresponding to a right ventricle, the target region R2 corresponding to a left ventricle, the target region R3 corresponding to a left atrium, and the target region R4 corresponding to a right atrium can be set (as shown in FIG. 4). Furthermore, in the heart T, the aggregated region R5 corresponding to a right side, the aggregated region R6 corresponding to a left side, the aggregated region R7 corresponding to the left and right ventricles, and the aggregated region R8 corresponding to the left and right atriums can be set (as shown in FIG. 5). The aggregated region R5 includes the target region R1 and the target region R4. The aggregated region R6 includes the target region R2 and the target region R3. The aggregated region R7 includes the target region R1 and the target region R2. The aggregated region R8 includes the target region R3 and the target region R4. As discussed above, each of the target regions R1 to R4 constitutes at least one of the aggregated regions R5 to R8.

In an ultrasound image obtained by image-capturing of the heart T, the target regions R1 to R4 and the aggregated regions R5 to R8 can be associated with respective different classifications. For example, classifications of “right ventricle”, “left ventricle”, “left atrium”, and “right atrium” can be respectively given to the target regions R1 to R4 as annotations, and classifications of “right”, “left”, “ventricle”, and “atrium” can be respectively given to the aggregated regions R5 to R8. Further, the classifications of “ventricle” and “atrium” can be respectively given to an aggregated region constituted by the target regions R1 and R2 and an aggregated region constituted by the target regions R3 and R4 as annotations.

FIG. 5 illustrates the aggregated regions R5 to R8 each of which is constituted by two target regions adjacent to each other. However, this is not limitative. For example, the aggregated region may be an aggregated region constituted by the right ventricle and the left atrium, an aggregated region constituted by the left ventricle and the right atrium, or an aggregated region constituted by three target regions.

The training data generating section 503 generates a training image in which the target regions R1 to R4 and the aggregated regions R5 to R8 are given with respective different classifications as annotations. The training image is training data that is used by the learning section 504 to carry out machine learning for the learning model. Note that the learning model is a model used by the detection section 102 of the information processing device 1 to detect the target regions and the aggregated regions.

The learning section 504 generates the learning model by machine learning. As discussed above, the learning model is generated by using the training image generated by the training data generating section 503.

### Process Executed by Identifying Section 103

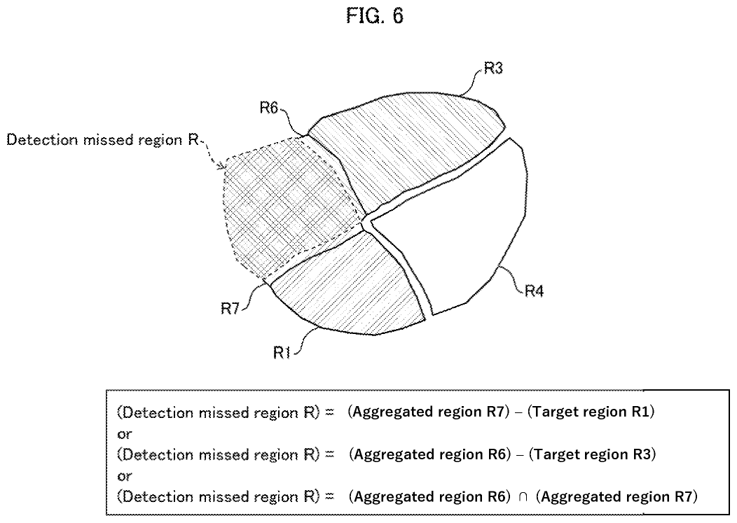

Next, the following will describe, with reference to FIGS. 6 to 9, a process executed by the identifying section 103. The following will provide an explanation by taking, as an example, a case where the detection section 102 identifies that a region constituting the aggregated regions R6 and R7 (i.e., the target region R2 corresponding to the left ventricle) is a detection missed region. The identifying section 103 identifies the detection missed region in the aggregated regions (e.g., the aggregated regions R6 and R7) detected by the detection section 102. FIG. 6 illustrates a state where the detection missed region R (e.g., the region corresponding to the target region R2) is identified in the aggregated regions having been detected.

The identifying section 103 carries out the identification by any one of the following processes (1) to (3):
(1) Identify, as the detection missed region, a region obtained by removing the detected target region R1 from the detected aggregated region R7.
(2) Identify, as the detection missed region, a region obtained by removing the detected target region R3 from the detected aggregated region R6.
(3) Identify, as the detection missed region, a region where the detected aggregated region R6 and the detected aggregated region R7 overlap each other.

In a result of detection of a target region and an aggregated region which detection is carried out by the detection section 102, an error(s) in their contours, sizes, and/or the like with respect to an actual target region and an actual aggregated region may possibly occur. For example, a case where the detected target region is smaller in size than the actual target region may occur. For another example, a case where the detected aggregated region is larger in size than the actual aggregated region may occur. In these cases, when the detected target region is simply removed from the detected aggregated region, a part of the aggregated region can remain in an area surrounding the target region having been removed. FIG. 7 is a view illustrating one example of a trouble which may occur in a case where a detection missed region is identified on the basis of a target region and an aggregated region having been detected. In a case where the detected target region R1 is removed from the detected aggregated region R7 without making any modification, a long, narrow region is identified between the contour of the aggregated region R7 and the contour of the target region R1, as illustrated in FIG. 7.

Thus, the identifying section 103 may identify, as the detection missed region, a region obtained by further removing, from the detected aggregated region, a region adjacent to an area surrounding the target region which is removed. The region adjacent to the area surrounding the target region which is removed may be a region increased when an expansion process is carried out, with use of a known image manipulation technique, on the target region which is removed. Note that the region adjacent to the area surrounding the target region which is removed may be a region increased when the target region which is removed is enlarged at a given magnification.

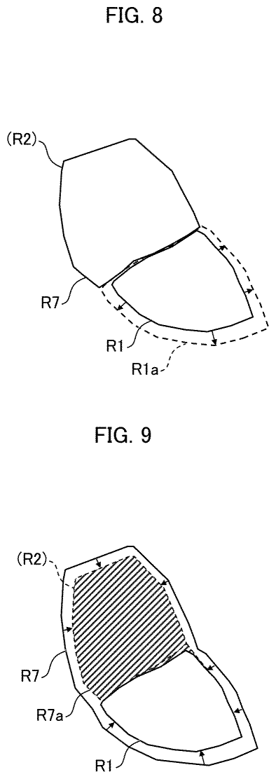

With this configuration, the information processing device 1 identifies, as the detection missed region, a region obtained by removing not only the detected target region but also the region adjacent to the area surrounding the detected target region from the detected aggregated region. Consequently, the information processing device 1 can reduce a possibility that a part of the aggregated region remains in the area surrounding the target region having been removed, thereby enhancing precision in identification of the detection missed region. FIG. 8 is a view illustrating one example of a method for identifying a detection missed region on the basis of target regions and aggregated regions having been detected. FIG. 8 illustrates a state of a process of removing, from the aggregated region R7, a region R1a obtained as a result of carrying out the expansion process with respect to the target region R1 which is removed.

In a result of detection of a target region and an aggregated region which detection is carried out by the detection section 102, an error(s) in their contours, sizes, and/or the like with respect to an actual target region and an actual aggregated region may possibly occur. For example, a case where the detected target region is smaller in size than the actual target region may occur. For another example, a case where the detected aggregated region is larger in size than the actual aggregated region may occur. Also in these cases, when the detected target region is simply removed from the detected aggregated region, a part of the aggregated region can remain in an area surrounding the target region having been removed.

Thus, the identifying section 103 may identify, as the detection missed region, a region obtained by removing the detected target region from a region yielded as a consequence of contraction on the detected aggregated region.

With this configuration, the information processing device 1 identifies, as the detection missed region, a region obtained by removing the detected target region from a region yielded as a consequence of contraction on the detected aggregated region. Consequently, the information processing device 1 can reduce a possibility that a part of the aggregated region remains in the area surrounding the target region having been removed, thereby enhancing precision in identification of the detection missed region. FIG. 9 is a view illustrating one example of a method for identifying a detection missed region on the basis of target regions and aggregated regions having been detected. FIG. 9 illustrates a state of a process of removing the target region R1 from a region R7a yielded as a consequence of a reduction process carried out on the aggregated region R7.

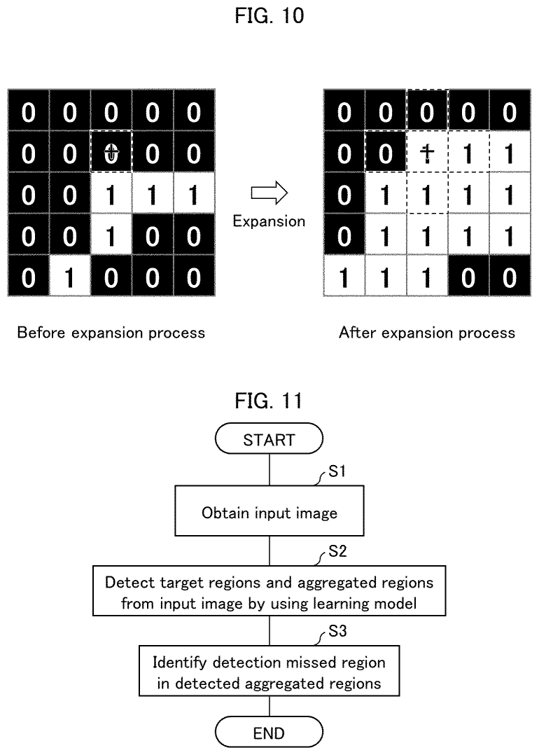

Example of Expansion Process and Reduction Process The description here will discuss, with reference to FIG. 10, an example of the expansion process and the reduction process involving use of morphology operation, the expansion process and the contraction process being executed by the identifying section 103. FIG. 10 is a view illustrating one example of the expansion process executed by the identifying section 103. The example illustrated in FIG. 10 will be explained by using, as an example, a process of expanding a white region (a region having a pixel value of 1) in an image whose pixel value has been binarized for simplification.

First, a kernel for scanning pixels in the image one by one is set. For example, in a case of a kernel having a size of 3 pixels×3 pixels, a center pixel can be called an “anchor”. In the example illustrated on the left side of FIG. 10, in a case of a kernel in which an anchor resides at a pixel (having a pixel value of “0” before expansion) in the second row and in the third column, a highest value among those of pixels each having a common side with (i.e., adjacent to) the anchor pixel is “1”. Thus, operation for changing the pixel value of the pixel corresponding to the position of the anchor from “0” to “1” is carried out. Such a process is carried out until the kernel scans the positions of all the pixels. The right side of FIG. 10 illustrates a result given when such an expansion process is carried out once.

In a case of a process of reducing a region (i.e., a white region) constituted by a pixel having a pixel value of “1”, operation inverse to the above is carried out. That is, in a case where the pixel value of the pixel where the anchor resides is “1” and a smallest value among those of pixels each having common side with (i.e., adjacent to) the anchor pixel is “0”, operation for changing the pixel value of the pixel corresponding to the position of the anchor to “0” is carried out.

The identifying section 103 may execute the above-described operation repeatedly until a part of the aggregated region does not remain in the area surrounding the target region having been removed. Consequently, the information processing device 1 can appropriately identify the detection missed region.

### Process Executed by Information Processing Device 1

Next, the following will describe, with reference to FIG. 11, a process executed by the information processing device 1. FIG. 11 is a flowchart illustrating one example of a flow of the process executed by the information processing device 1. Note that processes in the following steps may be executed by one or more information processing devices 1.

First, the image obtaining section 101 obtains an input image obtained by image-capturing of a heart T (step S1).

By using the learning model, the detection section 102 detects target regions and aggregated regions from the input image (step S2: detection step).

In a case where a detection result given by the detection section 102 includes a detection missed region, the identifying section 103 identifies the detection missed region in the detected aggregated regions (step S3: identification step). With this, even in a case where a target region having not been detected exists, the information processing device 1 can identify the detection missed region on the basis of the detected target regions and the detected aggregated regions. Consequently, the information processing device 1 can detect, from the input image obtained by image-capturing of the target object, the detection target region with high precision.

### Second Embodiment

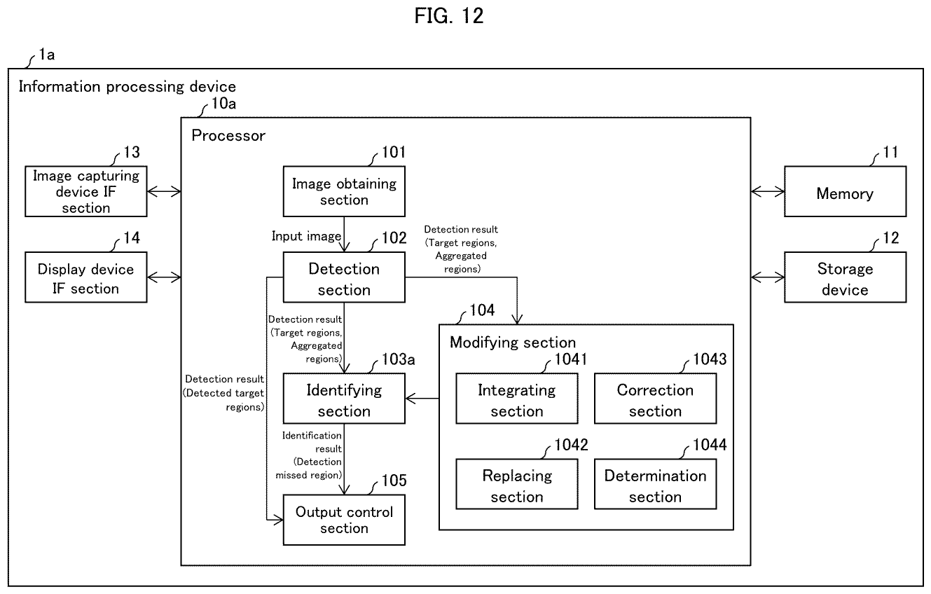

The following will describe, with reference to FIGS. 12 to 14, an information processing device la in accordance with another embodiment of the present invention. For convenience of description, any members of Second Embodiment that are identical in function to the members described for First Embodiment are assigned the same reference signs, and are not described again here.

### Information Processing Device 1a

The information processing device la has a function to modify a detection result of a target region. FIG. 12 is a block diagram illustrating one example of a configuration of a main part of the information processing device la in accordance with the present embodiment. A processor 10a included in the information processing device la can be realized by a logical circuit (hardware) formed in an integrated circuit (IC chip) or the like or can be realized by software. In a case where the processor 10a is realized by software, the processor 10a may be constituted by, for example, a CPU, a GPU, or a combination thereof. Further, in this case, the software is stored in the storage device 12. Then, the processor 10a loads the software into the memory 11 and executes the software.

The processor 10a functions as each of an image obtaining section 101, a detection section 102, an identifying section 103a, a modifying section 104, and an output control section 105. The following will describe the identifying section 103a and the modifying section 104.

The identifying section 103a identifies a detection missed region in detected aggregated regions, the detection missed region being a target region which has not been detected. Further, the identifying section 103a identifies a detection missed region indicated by a result of the later-described modification made by the modifying section 104. For example, the identifying section 103a may identify, as the detection missed region, a region that is included in an overlapping region where the detected aggregated regions overlap each other and that has not been detected as a target region. Also in a case where the overlapping region includes two or more target regions and a part of these target regions has been detected, the identifying section 103a can identify a detection missed region. Alternatively, the identifying section 103a may identify, as a detection missed region, a region obtained by removing the detected target region(s) from the detected aggregated region(s).

The modifying section 104 modifies erroneous detection made by the detection section 102. Typical examples of the erroneous detection made by the detection section 102 include the following (A) and (B).
(A) In a case where the target regions included in the target object is a prescribed number, a larger number of target regions than the predetermined number are detected. For example, the heart T has four target regions, that is, left and right ventricles and left and right atriums. Thus, in a case where more than four target regions are detected, erroneous detection is included.
(B) In a case where a positional relation between target regions included in the target object is determined, a plurality of target regions are detected as the same classification. For example, a relative positional relation between the four target regions, that is, the left and right ventricles and the left and right atriums of the heart T is determined. Thus, for example, in a case where two or more of the target regions are detected as the “right ventricle”, either of them is erroneous detection.

The modifying section 104 may include an integrating section 1041 and a replacing section 1042 that can modify the erroneous detection of the above-described example (A) and/or a correction section 1043 and a determination section 1044 that can modify the erroneous detection in the above-described example (B).

In a case where the number of target regions included in the target object is a prescribed number and a larger number of target regions than the prescribed number are detected, the integrating section 1041 carries out integration on, out of two combinations each made by some of the target regions having been detected, one combination including target regions overlapping each other to an overlapping degree which is highest and which exceeds a prescribed threshold, in such a manner that the target regions in the one combination are integrated into one target region. The degree of the overlapping may be a value obtained by dividing (i) an area of a region where the two target regions in the combination overlap each other by (ii) a sum of areas of the two target regions. Note that the prescribed threshold is a threshold having been set in advance so as to achieve a higher effect of modifying the erroneous detection.

According to this configuration, in a case where a larger number of target regions than the actual number of target regions included in the target object are detected, the information processing device la carries out integration on target regions in a combination which target regions overlap each other to a high overlapping degree, the integration being carried out such that the target regions in the combination are integrated into one target region. Consequently, in a case where a larger number of target regions more than the actual number are detected erroneously, the information processing device la can correct the detection result appropriately. For example, in a case where five target regions are detected from the input image obtained by image-capturing of the heart T, the integrating section 1041 can correct the erroneous detection by carrying out integration on, out of two combinations each made by some of the target regions having been detected, one combination including target regions overlapping each other to an overlapping degree which is highest and which exceeds a prescribed threshold, in such a manner that the target regions in the one combination are integrated into one target region.

The replacing section 1042 carries out replacement in a case where the number of detected target regions exceeds the prescribed number and a highest overlapping degree does not exceed the prescribed threshold, the replacement being carried out such that, among target regions having been detected as the same classification, a target region having a smallest area is replaced with an undetected region.

With this configuration, the information processing device 1a replaces, with an undetected region, one of the target regions having been erroneously detected as the same classification. Therefore, it is possible to correct the detection result.

For example, the integrating section 1041 carries out replacement in a case where five target regions are detected from the input image obtained by image-capturing of the heart T, the replacement being carried out such that, among the target regions having been detected as the same classification, a target region having a smallest area is replaced with an undetected region. As a result, this target region is a detection missed region, and is identified by the identifying section 103a. Therefore, it is possible to correct the erroneous detection.

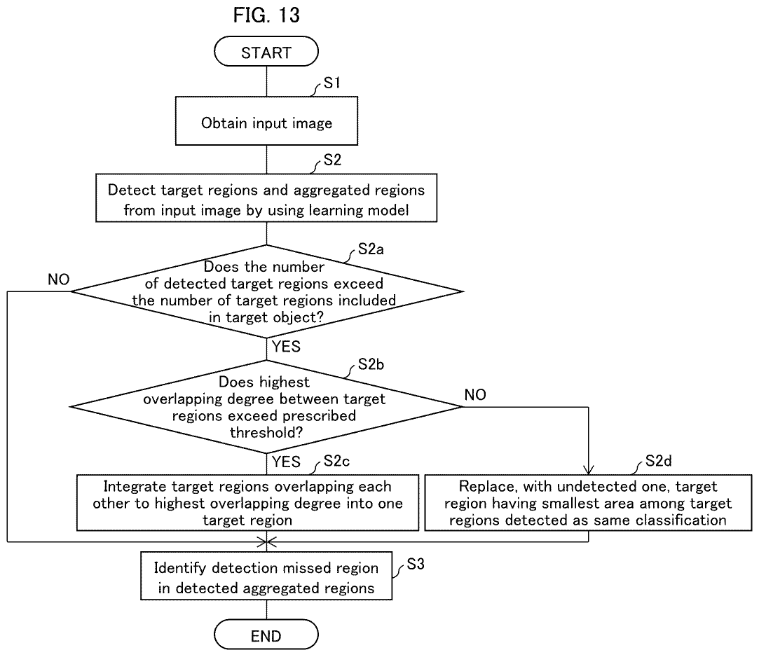

FIG. 13 is a flowchart illustrating one example of a flow of a process executed by the information processing device la including the integrating section 1041 and the replacing section 1042. Note that processes in the following steps may be executed by one or more information processing devices la.

In step S2a, the modifying section 104a determines whether or not the number of target regions having been detected exceeds the number of target regions included in the target object (for example, a heart). If YES in step S2a, the modifying section 104a determines whether or not the highest overlapping degree between the target regions exceeds the prescribed threshold (step S2b).

If YES in step S2b, the integrating section 1041 carries out integration on target regions in a combination which target regions overlap each other to a highest overlapping degree, in such a manner that the target regions are integrated into one target region (step S2c). Meanwhile, if NO in step S2b, the replacing section 1042 carries out replacement such that, among the target regions having been detected as the same classification, a target region having a smallest area is replaced with an undetected region (step S2d).

If NO in step S2a and steps S2c and S2d are already done, the identifying section 103a identifies the detection missed region (step S3: identification step).

With this configuration, the information processing device la can correct erroneous detection of a detection target. Therefore, it is possible to enhance precision in detection from the target object.

Referring back to FIG. 12, in a case where a positional relation between a plurality of target regions is determined, the correction section 1043 corrects classification of at least one of the plurality of target regions having been detected as the same classification, on the basis of a positional relation between the at least one of the plurality of target regions and the other one(s) of the target regions having been detected.

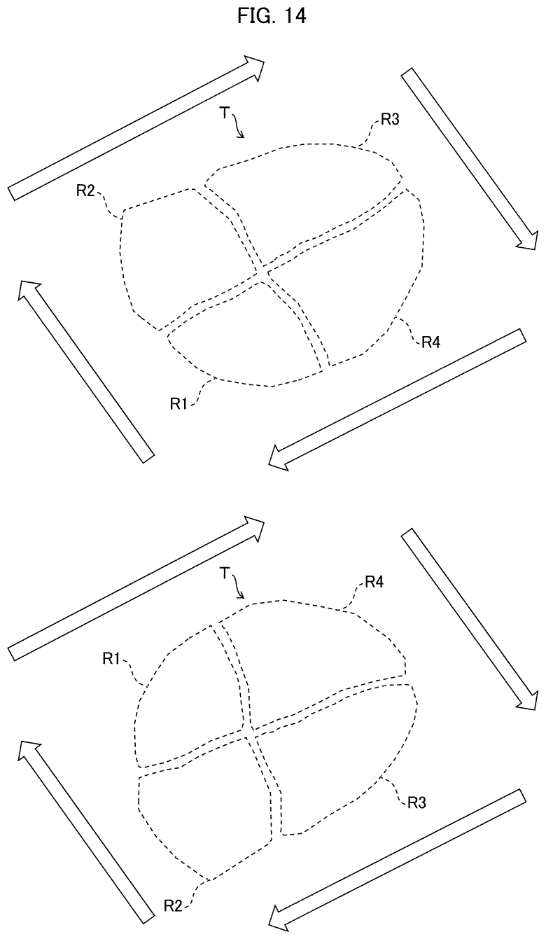

For example, a positional relation between the right ventricle, right atrium, left ventricle, and left atrium in the heart T is defined. For example, respective positions of the target regions differ between (i) an input image of the heart T captured from the abdominal side and (ii) an input image of the heart T captured from the back side. For example, in one of the input images shown in FIG. 14, the target regions R2, R3, and R4 are arranged clockwise in this order, starting from the target region R1. Meanwhile, in the other of the input images, the target regions R4, R3, and R2 are arranged clockwise in this order, starting from the target region R1. However, since the arrangement of the target regions in the target object is the same, the positional relations between the target regions in these input images are the same. That is, at least one of the plurality of target regions having been detected as the same classification is erroneous detection, and the classification of at least one of the plurality of target regions having been detected as the same classification can be corrected on the basis of a positional relation between the at least one of the plurality of target regions and the other one(s) of the target regions having been detected.

In a case where a positional relation between the target regions having been detected is determined, a position of a certain one of the detected target regions in the target object is uniquely determined on the basis of a positional relation between the certain one of the detected target regions and the other one(s) of the detected target regions. With this configuration, the information processing device la corrects classification of at least one of the plurality of target regions having been detected as the same classification, on the basis of a positional relation between the at least one of the plurality of target regions and the other one(s) of the target regions having been detected. Consequently, the information processing device la can correct inaccuracy in classification for at least one of the target regions having been detected, on the basis of a positional relation between the at least one of the target regions and the other one(s) of the target regions having been detected, and thus can modify the detection result.

In a state where all the target regions included in the target object have been detected, the determination section 1044 determines a positional relation between the target regions. Thanks to the configuration including the determination section 1044, the information processing device la can correctly determine the positional relation between the target regions included in the target object.

### Third Embodiment

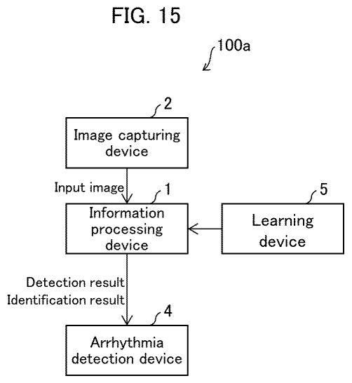

The following will describe, with reference to FIGS. 15 to 18, an information processing system 100 a in accordance with another embodiment of the present invention. For convenience of description, any members of Third Embodiment that are identical in function to the members described for First and Second Embodiments are assigned the same reference signs, and are not described again here.

### Information Processing System 100a

In a case where a moving image (ultrasonic moving image) including images of a plurality of frames obtained by image-capturing of a heart T is used as the input image, the information processing device 1 can output, for each of the plurality of frames, a detection result including target regions having been detected and a detection missed region having been identified. As illustrated in FIG. 15, an information processing system 100a includes an arrhythmia detection device 4 that detects arrhythmia on the basis of time-series changes in an area of each of the target regions having been detected and the detection missed region having been identified, each of the target regions and the detection missed region being obtained by the information processing device 1. The arrhythmia detection device 4 detects arrhythmia on the basis of time-series changes in the area of each of the left atrium, right atrium, left ventricle, and right ventricle having been detected or having been identified as a detection missed region(s) in each of the frames of the ultrasonic moving image obtained by image-capturing of the heart T in the information processing device 1. With the above configuration, the arrhythmia detection device 4 can precisely determine arrhythmia.

### Process Executed by Arrhythmia Detection Device

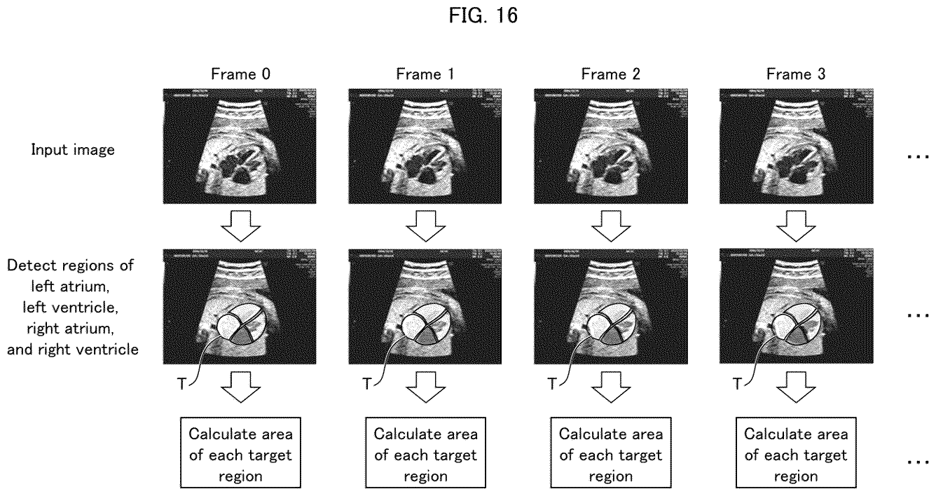

The following will describe, with reference to FIG. 16, a process executed by the information processing system 100a including the arrhythmia detection device 4. FIG. 16 is a view illustrating one example of a process executed by the information processing system 100a.

The uppermost row in FIG. 16 shows images corresponding to respective frames 0, 1, 2, 3, . . . of the input image obtained by image-capturing of a fetus's heart. The middle row in FIG. 16 shows the images of the respective frames superimposed with the target regions indicating the left atrium, right atrium, left ventricle, and right ventricle having been detected by the information processing device 1 or having been identified as a detection missed region(s). The lowermost row in FIG. 16 shows states in which areas of the left atrium, right atrium, left ventricle, and right ventricle having been detected or having been identified as a detection missed region(s) by the information processing device 1 are determined in each frame. The information processing device 1 executes processes from the uppermost row to the middle row in FIG. 16, and the arrhythmia detection device 4 executes processes from the middle row to the lowermost row in FIG. 16.

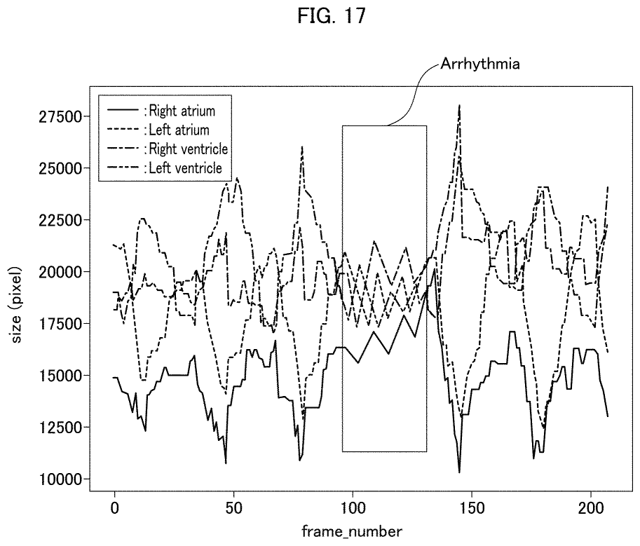

Next, the following will describe, with reference to FIG. 17, a process in which the arrhythmia detection device 4 detects arrhythmia. FIG. 17 is a view illustrating one example of the process in which the arrhythmia detection device 4 detects arrhythmia. In FIG. 17, the areas (sizes) of the left atrium, right atrium, left ventricle, and right ventricle having been detected or having been identified as a detection missed region(s) by the information processing device 1 are represented by graphs. A unit of each of the areas may be mm², for example. Alternatively, each of the areas may be represented by the number of pixels included in the region corresponding to a respective one of the left atrium, right atrium, left ventricle, and right ventricle in the image. In a case where arrhythmia does not occur, cyclic increasing and decreasing patterns are repeated in the areas (sizes) of the left atrium, right atrium, left ventricle, and right ventricle. In contrast to this, in a case where arrhythmia occurs, the cyclic increasing and decreasing patterns are disordered, as observed in the frames indicated by the quadrangle frame shown in FIG. 17. The arrhythmia detection device 4 can detect arrhythmia by detecting disorders in the cyclic increasing and decreasing patterns of the areas (sizes) of the left atrium, right atrium, left ventricle, and right ventricle.

### Process Executed by Information Processing Device and Arrhythmia Detection Device

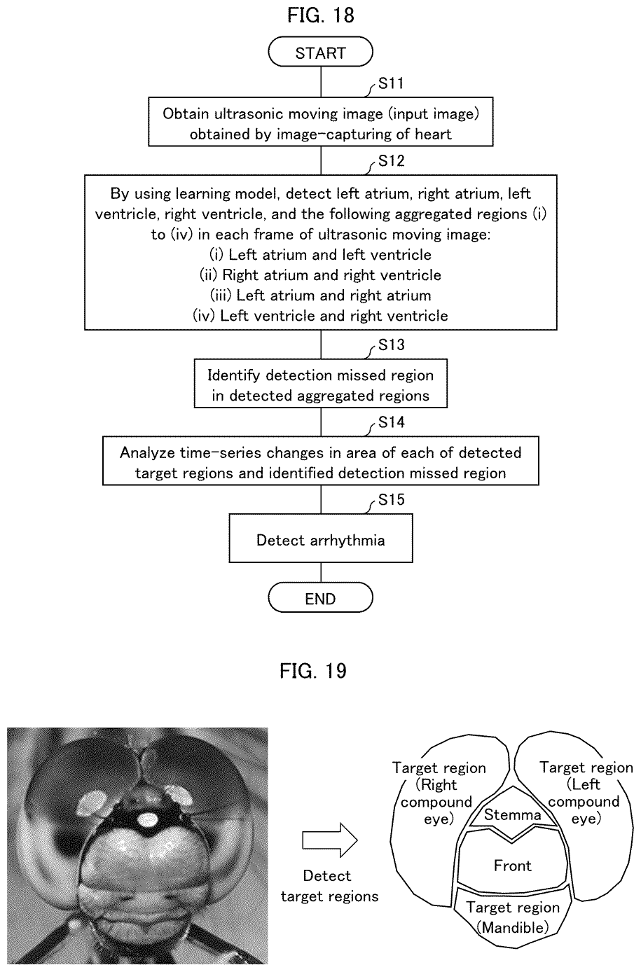

Next, the following will describe, with reference to FIG. 18, a process executed by the information processing device 1 and the arrhythmia detection device 4. FIG. 18 is a flowchart illustrating one example of a process executed by the information processing system 100a. Note that processes in the following steps may be executed by one or more information processing devices 1 and one or more arrhythmia detection devices 4.

The image obtaining section 101 of the information processing device 1 obtains an ultrasonic moving image obtained by image-capturing of a heart (step S11). Then, the detection section 102 of the information processing device 1 uses a learning model to detect the left atrium, the right atrium, the left ventricle, the right ventricle, and the following aggregated regions (i) to (iv) in each of the frames of the ultrasonic moving image (step S12).
(i) The left atrium and the left ventricle.
(ii) The right atrium and the right ventricle.
(iii) The left atrium and the right atrium.
(iv) The left ventricle and the right ventricle.

Next, the identifying section 103 identifies a detection missed region in the detected aggregated regions (step S13).

Subsequently, the arrhythmia detection device 4 analyzes time-series changes in the area of each of the target regions having been detected and the detection missed region(s) having been identified, the area of each of the target regions and the detection missed region(s) being obtained by the information processing device 1 (step S14), and detects arrhythmia (step S15).

### Fourth Embodiment

### Target Regions in Dragon Fly's Head

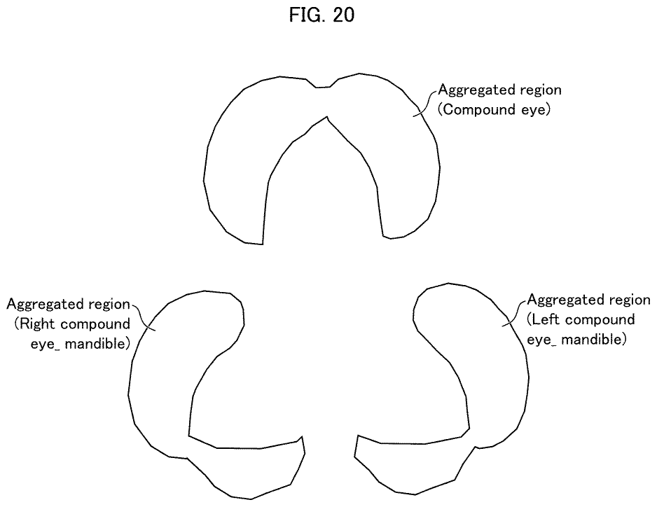

The following will describe, with reference to FIGS. 19 and 20, a case where the target object is a dragon fly's head. FIG. 19 is a view illustrating an example of target regions in an image of a dragon fly's head, captured from front of the dragon fly's head. FIG. 20 is a view illustrating an example of aggregated regions in the image of the dragon fly's head, captured from the front of the dragon fly's head. The left side of FIG. 19 illustrates the image obtained by image-capturing of the dragon fly's head. Each of the information processing devices 1 and la in accordance with the present invention is capable of detecting a target region in an input image obtained by image-capturing of a dragon fly's head and identifying a detection missed region.

In a case where regions corresponding to a right compound eye, a left compound eye, and a mandible shown in the right side of FIG. 19 are target regions, three regions illustrated in FIG. 20 may be aggregated regions.

Each of the information processing devices 1 and la is capable of detecting, with high precision, the target regions respectively corresponding to anatomical organs in the dragon fly. As discussed above, the information processing devices 1 and la are applicable to detection of target regions in various kinds of target objects. Therefore, the information processing devices 1 and la are applicable to, for example, generation of a learning material regarding an anatomical structure of a living organism, the learning material using, as a material, an input image obtained by image-capturing of the living organism.

### Software Implementation Example

The functions of the information processing device 1, 1a (hereinafter, referred to as “device”) can be realized by a program for causing a computer to function as the device, the program causing the computer to function as the control blocks (in particular, the sections included in the processor 10, 10a) of the device.

In this case, the device includes, as hardware for executing the program, a computer that includes at least one control device (e.g., a processor) and at least one storage device (e.g., a memory). The functions described in the foregoing embodiments are realized by executing the program with use of the control device and the storage device.

The program may be recorded in one or more non-transitory computer-readable storage media. The one or more storage media may or may not be included in the device. In the latter case, the program may be made available to the device via any wired or wireless transmission medium.

Further, some of or all of the functions of the control blocks can be realized by a logic circuit. For example, the present invention encompasses, in its scope, an integrated circuit in which a logic circuit that functions as each of the control blocks is formed. In addition, the functions of the control blocks can also be implemented by, for example, a quantum computer.

The processes described in the above embodiments may be carried out by artificial intelligence (AI). In this case, the AI may operate in the control device, or may operate in another device (e.g., an edge computer or a cloud server).

The present invention is not limited to the embodiments, but can be altered by a skilled person in the art within the scope of the claims. The present invention also encompasses, in its technical scope, any embodiment derived by combining technical means disclosed in differing embodiments.

Aspects of the present invention can also be expressed as follows:

An information processing device in accordance with a first aspect of the present invention includes: a detection section that detects, from an input image obtained by image-capturing of a target object, target regions included in the target object and aggregated regions each constituted by two or more of the target regions, the detection being carried out by using a learning model having been trained using a training image in which the target regions and the aggregated regions are given with respective different classifications as annotations; and an identifying section that identifies a detection missed region in the aggregated regions having been detected, the detection missed region being a target region which has not been detected.

An information processing device in accordance with a second aspect of the present invention may be configured such that, in the first aspect, each of the target regions constitutes at least one of the aggregated regions.

An information processing device in accordance with a third aspect of the present invention may be configured such that, in the first or second aspect, the identifying section identifies, as the detection missed region, a region which is included in an overlapping region where some of the aggregated regions having been detected overlap each other and which has not been detected as a target region.

An information processing device in accordance with a fourth aspect of the present invention may be configured such that, in any one of the first to third aspects, the identifying section identifies, as the detection missed region, a region obtained by removing, from at least one of the aggregated regions having been detected, at least one of the target regions

An information processing device in accordance with a fifth aspect of the present invention may be configured such that, in the fourth aspect, the identifying section identifies, as the detection missed region, a region obtained by further removing, from the at least one of the aggregated regions having been detected, a region adjacent to an area surrounding the at least one of the target regions which is removed.

An information processing device in accordance with a sixth aspect of the present invention may be configured such that, in the fourth or fifth aspect, the identifying section identifies, as the detection missed region, a region obtained by removing, from a region yielded as a consequence of contraction on at least one of the aggregated regions, at least one of the target regions having been detected.

An information processing device in accordance with a seventh aspect of the present invention may be configured such that, in any one of the first to sixth aspects, the information processing device further comprises an integrating section that carries out integration in a case where the number of the target regions included in the target object is a prescribed number and a larger number of target regions than the prescribed number are detected, the integration being carried out on, out of two combinations each made by some of the target regions having been detected, one combination including target regions overlapping each other to an overlapping degree which is highest and which exceeds a prescribed threshold, in such a manner that the target regions in the one combination are integrated into one target region.

An information processing device in accordance with an eighth aspect of the present invention may be configured such that, in the seventh aspect, the degree of the overlapping is a value obtained by dividing (i) an area of a region where two target regions in the one combination overlap each other by (ii) a sum of areas of the two target regions.

An information processing device in accordance with a ninth aspect of the present invention may be configured such that, in the seventh or eighth aspect, the information processing device further includes a replacing section that carries out replacement in a case where the number of the target regions having been detected exceeds the prescribed number and the highest overlapping degree does not exceed the prescribed threshold, the replacement being carried out such that, among target regions having been detected as a same classification, a target region having a smallest area is replaced with an undetected region.

An information processing device in accordance with a tenth aspect of the present invention may be configured such that, in any one of the first to ninth aspects, a positional relation between the target regions is determined; and the information processing device further comprises a correction section that corrects classification of at least one of a plurality of target regions having been detected as a same classification, on a basis of a positional relation between (i) the at least one of the plurality of target regions having been detected as the same classification and (ii) another one or the other ones of the target regions having been detected.

An information processing device in accordance with an eleventh aspect of the present invention may be configured such that, in the tenth aspect, the information processing device further includes a determination section that determines, in a state where all the target regions included in the target object have been detected, a positional relation between the target regions.

The target object is a heart; the target regions are a left atrium, a right atrium, a left ventricle, and a right ventricle; the input image is an ultrasonic moving image; and an arrhythmia detection device in accordance with a twelfth aspect of the present invention detects arrhythmia on a basis of time-series changes in an area of each of the target regions having been detected and the detection missed region having been identified, each of the target regions and the detection missed region being obtained in each of frames of the input image by an information processing device in accordance with any one of the first to eleventh aspects.

A region identification method in accordance with a thirteenth aspect of the present invention is a region identification method that is executed by one or more information processing devices, the region identification method including: a detection step of detecting, from an input image obtained by image-capturing of a target object, target regions included in the target object and aggregated regions each constituted by two or more of the target regions, the detection being carried out by using a learning model having been trained using a training image in which the target regions and the aggregated regions are given with respective different classifications as annotations; and an identification step of identifying a detection missed region in the aggregated regions having been detected, the detection missed region being a target region which has not been detected.

A region identification program for causing a computer to function as an information processing device in accordance with the first aspect, the region identification program causing the computer to function as the detection section and the identifying section.

### REFERENCE SIGNS LIST

1, 1a: information processing device
2: image capturing device
3: display device
4: arrhythmia detection device
100, 100a: information processing system
102: detection section
103, 103a: identifying section
1041: integrating section
1042: replacing section
1043: correction section
1044: determination section
S2: detection step
S3: identification step

1.

1. An information processing device comprising:
a detection section that detects, from an input image obtained by image-capturing of a target object, target regions included in the target object and aggregated regions each constituted by two or more of the target regions, the detection being carried out by using a learning model having been trained using a training image in which the target regions and the aggregated regions are given with respective different classifications as annotations; and
an identifying section that identifies a detection missed region in the aggregated regions having been detected, the detection missed region being a target region which has not been detected.

2. The information processing device according to claim 1, wherein:
each of the target regions constitutes at least one of the aggregated regions.

3. The information processing device according to claim 1, wherein:
the identifying section identifies, as the detection missed region, a region which is included in an overlapping region where some of the aggregated regions having been detected overlap each other and which has not been detected as a target region.

4. The information processing device according to claim 1, wherein:
the identifying section identifies, as the detection missed region, a region obtained by removing, from at least one of the aggregated regions having been detected, at least one of the target regions having been detected.

5. The information processing device according to claim 4, wherein:
the identifying section identifies, as the detection missed region, a region obtained by further removing, from the at least one of the aggregated regions having been detected, a region adjacent to an area surrounding the at least one of the target regions which is removed.

6. The information processing device according to claim 4, wherein:
the identifying section identifies, as the detection missed region, a region obtained by removing, from a region yielded as a consequence of contraction on at least one of the aggregated regions, at least one of the target regions having been detected.

7. The information processing device according to claim 1, further comprises:
an integrating section that carries out integration in a case where the number of the target regions included in the target object is a prescribed number and a larger number of target regions than the prescribed number are detected, the integration being carried out on, out of two combinations each made by some of the target regions having been detected, one combination including target regions overlapping each other to an overlapping degree which is highest and which exceeds a prescribed threshold, in such a manner that the target regions in the one combination are integrated into one target region.

8. The information processing device according to claim 7, wherein:
the degree of the overlapping is a value obtained by dividing (i) an area of a region where two target regions in the one combination overlap each other by (ii) a sum of areas of the two target regions.

9. The information processing device according to claim 7, further comprising:
a replacing section that carries out replacement in a case where the number of the target regions having been detected exceeds the prescribed number and the highest overlapping degree does not exceed the prescribed threshold, the replacement being carried out such that, among target regions having been detected as a same classification, a target region having a smallest area is replaced with an undetected region.

10. The information processing device according to claim 1, wherein:
a positional relation between the target regions is determined; and
the information processing device further comprises a correction section that corrects classification of at least one of a plurality of target regions having been detected as a same classification, on a basis of a positional relation between (i) the at least one of the plurality of target regions having been detected as the same classification and (ii) another one or the other ones of the target regions having been detected.

11. The information processing device according to claim 10, further comprising:
a determination section that determines, in a state where all the target regions included in the target object have been detected, a positional relation between the target regions.

12. An arrhythmia detection device, wherein:
the target object is a heart;
the target regions are a left atrium, a right atrium, a left ventricle, and a right ventricle;
the input image is an ultrasonic moving image; and
the arrhythmia detection device detects arrhythmia on a basis of time-series changes in an area of each of the target regions having been detected and the detection missed region having been identified, each of the target regions and the detection missed region being obtained in each of frames of the input image by an information processing device recited in claim 1.

13. A region identification method that is executed by one or more information processing devices, the region identification method comprising:
a detection step of detecting, from an input image obtained by image-capturing of a target object, target regions included in the target object and aggregated regions each constituted by two or more of the target regions, the detection being carried out by using a learning model having been trained using a training image in which the target regions and the aggregated regions are given with respective different classifications as annotations; and
an identification step of identifying a detection missed region in the aggregated regions having been detected, the detection missed region being a target region which has not been detected.

14. A non-transitory computer-readable storage medium storing region identification program for causing a computer to function as an information processing device recited in claim 1, the region identification program causing the computer to function as the detection section and the identifying section by executing the region identification program with a processor of the computer.
